(19) United States
(12) Patent Application Publication
(10) Pub. No.: US 2015/0297815 A1
(43) Pub. Date: Oct. 22, 2015

(54) METHOD OF ADSORPTION OF IONS FROM A DIALYSIS FLUID

(71) Applicant: TRIOMED AB, LUND (SE)

(72) Inventor: Nina MEINANDER, SÖDRA SANDBY (SE)

(73) Assignee: TRIOMED AB, LUND (SE)

(21) Appl. No.: 14/646,789

(22) PCT Filed: Nov. 22, 2013
(86) PCT No.: PCT/SE2013/000182

§ 371 Date: May 22, 2015

(30) Foreign Application Priority Data
Nov. 23, 2012 (SE) 1230132-1

(51) Int. Cl. A61M 1/16; A61M 1/28

(52) U.S. Cl. CPC A61M 1/1694; A61M 1/28

(57) ABSTRACT

A method of adsorption of potassium ions from a peritoneal dialysis fluid, which has been removed from the peritoneal cavity of a patient performing peritoneal dialysis for subsequent recirculation and introduction into the peritoneal cavity of the patient. The peritoneal dialysis fluid is passed through a potassium adsorbent, such as a zeolite, which has been pre-equilibrated with an equilibration fluid comprising Na⁺, Ca²⁺, Mg²⁺, Cl⁻, lactate, in substantially the same concentration as in the peritoneal dialysis fluid and which equilibration fluid lacks K⁺. The zeolite may be titrated with an acid. Alternatively, a zeolite having a Si:Al ratio which is larger than 5:1 is used. Alternatively, a cation exchange material, such as sodium polystyrene sulfonate, is used.

### FIELD OF INVENTION

The present invention relates to an adsorbent intended for use during dialysis. More specifically, the invention relates to a method of removing potassium by adsorption during dialysis with recirculation and regeneration of the dialysis fluid.

### BACKGROUND

Patients suffering from end-stage renal disease need dialysis treatment, either during the rest of their lives or until transplantation of a kidney. Dialysis treatment may also be used at other diseases or indications, such as poisoning or high levels of trace minerals.

During dialysis, large quantities of dialysate may be consumed. The spent dialysate is normally discarded.

In order to reduce the amount of used fluid, the spent dialysis fluid may be regenerated and recirculated. In dialysis, the regeneration should address three main areas or objects, namely 1) removal of urea, 2) removal of creatinine or organic metabolites and other waste products, and 3) balancing of electrolyte ions. In addition, 4) water should normally be removed from the patient.

Most dialysate regeneration systems use activated carbon, which is effective in removing a great number of different waste products or organic metabolites, including creatinine and uric acid. However, activated carbon is not effective in removing urea or balancing electrolyte ions.

The present invention is directed to the third object mentioned above, namely balancing of electrolyte ions.

In previous dialysis, both hemodialysis and peritoneal dialysis, the potassium ion has not been problematic, since sufficient potassium ion removal is achieved when the dialysate containing surplus potassium ions is discarded.

In peritoneal dialysis using regeneration and recirculation of the dialysis fluid, only a small amount of the dialysis fluid is discarded as surplus fluids, while most of the spent fluid containing among others potassium ions is recirculated back to the patient after regeneration. In this case, removal of potassium ions may be an issue, which is a new problem in dialysis fluid regeneration.

Removal of potassium ions from the recirculated dialysis fluid should take place without substantially influencing the concentration of other ions in the peritoneal dialysis fluid and without altering the pH of the peritoneal dialysis fluid.

### SUMMARY OF THE INVENTION

Accordingly, an object of the present invention is to mitigate, alleviate or eliminate one or more of the above-identified deficiencies and disadvantages singly or in any combination.

In an aspect, there is provided a method of adsorption of ions from a peritoneal dialysis fluid, which has been removed from the peritoneal cavity of a patient performing peritoneal dialysis for subsequent recirculation and re-introduction into the peritoneal cavity of the patient, or from a hemo-dialysis fluid which has been removed from a dialyzer for subsequent recirculation and re-introduction into the dialyzer; characterized by the step of: contacting the removed dialysis fluid with a potassium adsorbent for adsorption of potassium ions.

The potassium adsorbent may be an ion exchange material, which adsorbs potassium ions and releases sodium ions in their place. The amount of dialysis fluid may be less than 10 liters and that the dialysis fluid is discarded at least once per day, for example less than 5 liters, such as less than 3 liters.

The potassium adsorbent is a sodium polystyrene sulfonate ion exchange material. In an embodiment, binding of calcium ions and magnesium ions by the adsorbent is compensated by oral intake or by addition of calcium ions and magnesium ions to the recirculated fluid, downstreams of the ion exchanger.

In another embodiment, the potassium adsorbent is a zeolite, which has been pre-equilibrated with an equilibration fluid comprising at least one of: Na⁺, Ca²⁺, Mg²⁺, Cl⁻, lactate, bicarbonate and glucose, which equilibration fluid lacks K. The equilibration fluid may contain: Na⁺, Ca²⁺, Mg²⁺, Cl⁻ and lactate, in substantially the same concentration as in peritoneal dialysis fluid.

In a further embodiment, the zeolite has a Si:Al ratio which is larger than 5:1.

In a yet further embodiment, the zeolite has a Si:Al ratio which is larger than 1:1 and wherein the zeolite during or after the pre-equilibration is titrated to near neutral pH using acid. The acid may be added to the equilibration fluid so that said fluid before or during the pre-equilibration has a pH of less than 5.0.

### BRIEF DESCRIPTION OF THE DRAWINGS

Further objects, features and advantages of the invention will become apparent from the following detailed description of embodiments of the invention with reference to the drawings, in which:

FIG. 1 is a schematic diagram of a system in which the zeolite may be used.

### DETAILED DESCRIPTION OF EMBODIMENTS

Below, several embodiments of the invention will be described. These embodiments are described in illustrating purpose in order to enable a skilled person to carry out the invention and to disclose the best mode. However, such embodiments do not limit the scope of the invention. Moreover, certain combinations of features are shown and discussed. However, other combinations of the different features are possible within the scope of the invention.

In previously known dialysis systems, urea may be removed by means of urease, which is an enzyme catalyzing the conversion of urea to ammonium and carbonate ions. As described in the document EP0046971A1, the ammonium cations, or ammonium, or ammonia may be removed by an ion-exchange medium that is selective towards ammonium. The carbonate is precipitated as calcium carbonate or calcium bicarbonate.

There are alternatives to the urease system, for example as disclosed in document WO 2010/141942A2.

Dialysis may take place in two principally different manners, namely hemodialysis and peritoneal dialysis.

In hemodialysis, blood is removed from the patient into an extracorporeal circuit comprising a dialyzer. The dialyzer comprises a semipermeable membrane dividing the dialyzer into a blood compartment and a dialysate compartment. Blood is passed through the extracorporeal circuit to the blood compartment and into contact with one side of the membrane and further back to the patient. A dialysis fluid is passed into the dialysate compartment and into contact with the other side of the membrane. The membrane comprises pores having a size, which will prevent large molecules to pass through the pores while smaller molecules may pass freely through the membrane via the pores. The cut-off size of the pores is normally expressed as the size of the molecule (or ion) that can pass. A common cut-off size is about 2000 Dalton, which allows smaller molecules to pass. Another common cut-off size is about 20.000 Dalton, which allows small and middle-size molecules to pass. In both cases albumin having a size of 58.000 Dalton and larger molecules may not pass through the membrane.

The dialysis fluid passing inside the dialysis compartment of the dialyzer has a specific composition, which partly mimics the composition of the blood. If there is a concentration gradient of a substance between blood and the dialysis fluid, such substance will tend to pass to the fluid having the lower concentration. For example, blood comprises urea and the dialysis fluid does not, whereby urea (a small molecule) passes from blood through the pores of the membrane and to the dialysis fluid. On the other hand, the dialysis fluid may comprise bicarbonate in a concentration, which is larger than the bicarbonate concentration of blood, whereby bicarbonate is transferred from the dialysis fluid through the pores of the membrane and into the blood.

By tailoring the composition of the dialysis fluid, metabolic waste products, such as urea and creatinine, are removed from the blood, and substances, such as bicarbonate, is added to the blood, while other substances, such as sodium, potassium, calcium and magnesium ions are balanced, meaning that there is a net addition to—or removal from—the blood depending on the relative concentrations.

In addition, some water is removed from the blood of the patient, for example by exerting a small negative pressure over the membrane causing ultrafiltration.

There are variants of the hemodialysis method, such as hemofiltration and hemodiafiltration.

A commonly used hemodialysis fluid may comprise the following ions: sodium, potassium, calcium, magnesium, chloride and bicarbonate.

In addition, the pH of the dialysis fluid should be close to physiological pH of 7.4, for example between 6.0 and 7.6. If the pH of the dialysis fluid is too high, there is a risk of precipitation of calcium carbonate, which should be avoided.

In peritoneal dialysis, the peritoneal membrane of the patient is used in place of the dialyzer membrane. A peritoneal dialysis fluid is instilled in the peritoneal cavity of the patient and exchange of substances between the blood and the peritoneal dialysis fluid may take place over the peritoneal membrane similar to hemodialysis. No extracorporeal circuit for blood is required, which is a considerable advantage. However, the peritoneal membrane may be smaller than the dialyzer membrane, resulting in less efficient dialysis. Thus, peritoneal dialysis is traditionally used for patients having some residual kidney function.

A drawback is that the peritoneal membrane is sensitive to certain substances and bacteria, which may cause peritonitis—an inflammation of the peritoneal membrane and adjacent tissue.

It is not possible to exert a negative pressure in the peritoneal cavity and another mechanism is required for fluid removal. Thus, an osmotically or oncotically active substance is added to the peritoneal dialysis fluid to cause fluid removal via osmosis. A commonly used substance is glucose or another carbohydrate.

Thus, a peritoneal dialysis fluid may comprise the following substances: sodium chloride, calcium chloride, magnesium chloride, and at least one of sodium acetate, sodium lactate, sodium bicarbonate. In addition, the fluid comprises an osmotic or oncotic agent such as glucose for excess fluid removal.

It is important that the pH is close to physiological pH in order to prevent any harm or discomfort to the patient. Normally, the pH should be between 6.0 and 7.5.

Regeneration and recirculation of dialysate enables a great reduction in the volume of dialysis fluid used per day. This is a prerequisite for creating a portable/wearable dialysis system which is not connected to a supply of fresh water or heavy stationary equipment in hemodialysis and allows the patient freedom from frequent exchanges of peritoneal dialysis fluid in peritoneal dialysis.

In a hemodialysis system, the regeneration circuit may be arranged as shown in the document GB1484642A, which discloses a system comprising urease for catalytic conversion of urea to ammonium and carbonate ions. This conversion results in an increase of the pH of the solution to greater than pH=8.5, which may be lowered by addition of an acid. As acids carbon dioxide can be used as well as a cation exchanger HZ, which functions as a weak acid. The ammonium ions are adsorbed by a zeolite, such as phillipsite loaded with sodium ions NaZ. Calcium ions are added after the phillipsite column to precipitate the carbonate ion. It is specifically mentioned that the zeolites removed less potassium ions from a fluid containing 4.7 mmol/L K⁺, than zirconium phosphate, another ion exchange material used in regeneration of dialysis fluid.

In a peritoneal system with regeneration of dialysis fluid, most of the fluid, for example about 2 liters, may reside inside the peritoneal cavity of the patient and less than one liter may be present in the system outside the patient.

In patients with renal failure it is highly important to keep the blood potassium ion concentration within narrow physiological limits, to avoid hyper- or hypokalemic conditions, which could be potentially life-threatening. In the systems used today, where the dialysis fluid is discarded, removal of excess potassium ions from the patient's blood is seldom a problem. In hemodialysis, the dialysis fluid normally comprises a low concentration of potassium ions of for example 2 mM (millimole per liter), which will ensure that adequate potassium concentration is maintained in the patient.

In peritoneal dialysis, the potassium ion concentration of the peritoneal dialysis fluid is normally zero, but a low concentration of potassium ions may be added to prevent severe hypokalemia.

It is reported that about 0.8% of CAPD patients have hyperkalemia while 10%-15% of CAPD patients require potassium-salt supplementation for hypokalemia.

Thus, too small removal of potassium ions is not regarded as a problem in conventional peritoneal dialysis.

Patients with hyperkalemia may ingest a potassium-adsorbing agent, for example Resonium, which is a calciumpolystyrensulfonate. The potassium-adsorbing agent binds potassium in the intestine.

In a system where dialysis fluid is regenerated and recirculated, potassium ions need to be continuously removed from the dialysis fluid in sufficient amounts to keep the blood potassium level normal in order to counteract or balance intake of potassium via the food.

It is normally desired to remove from 0 to 1.2 grams potassium per day, corresponding to 0 to 30 mmoles per day. Blood normally comprises 3.5 to 5.0 mM potassium.

In a peritoneal dialysis fluid of 3 liters without potassium ions, and assuming complete equilibration with blood potassium levels, up to 15 mmole potassium ions are removed by the fluid itself. Thus, another 15 mmoles should be removed per day. If still lower volumes of peritoneal fluid is used, such as 1.5 liters, up to 23 mmole per day may be removed per day.

Peritoneal dialysis fluid additionally comprises sodium, calcium, magnesium, lactate and chloride ions, and glucose or another carbohydrate. The removal of potassium ions should take place without substantially influencing the other components of the peritoneal dialysis fluid.

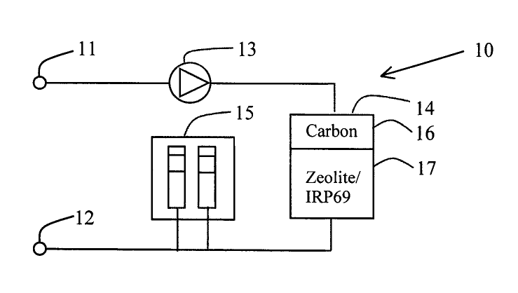

FIG. 1 is a schematic diagram of an embodiment of a regeneration device. The regeneration device 10 comprises an inlet 11 for a dialysis fluid and an outlet 12 for regenerated dialysis fluid. The inlet and outlet may be connected to a dual lumen peritoneal dialysis catheter installed in a patient. The peritoneal cavity of the patient comprises peritoneal dialysis fluid, which should be regenerated with the adsorption device of FIG. 1.

Alternatively, the inlet 11 and outlet 12 may be connected to a dialyzer for hemodialysis, hemofiltration or hemodiafiltration.

From the inlet 11, the fluid passes via a line to a pump 13, which may be a peristaltic pump. From the pump, the fluid passes to an adsorption cartridge 14. From the cartridge 14, the fluid passes via a line to the outlet 12. A cartridge 15 comprising one or several replacement solutions may be arranged to add replacement solutions to the outgoing dialysis fluid. So far, the regeneration device 10 is similar to previously known technique.

As is also conventional, the cartridge 14 may comprise several adsorbents. One adsorbent, which is included in almost every regeneration systems, is a first adsorbent 16 comprising activated carbon.

In addition, there is a potassium adsorbent 17, which is effective in the adsorption of potassium. There may be further adsorbents included in the adsorbent cartridge.

A zeolite is an adsorbent, which is suitable for adsorption of potassium ions through the mechanism of ion-exchange. Zeolites have been used before in dialysis for adsorption of ammonium ions as described in the above-mentioned document GB1484642A and are shown to be non-toxic and chemically stable in a dialysis fluid environment.

Zeolites are aluminosilicates, which exist as natural minerals and can also be made synthetically. Zeolites form a regular microporous crystal framework composed of TO₄ (T=Si/Al), in which the ratio (Si+Al):O equals ½. Nearly 200 different zeolite structures, of which 40 occur as natural minerals, have been described. The aluminosilicate structure is negatively charged. This requires the presence of extra-framework positively charged cations within the zeolite structure to keep the overall framework neutral. These cations can partly be exchanged with cations in the surrounding fluid depending on their location within the crystal framework. Zeolites have large vacant spaces or cages in their structures that accommodate cations such as for example sodium, potassium, barium and calcium and even small molecules, such as water, and polyatomic cations, such as ammonium. The affinity for a specific ion is affected by the pore size and crystal framework of the zeolite. Thus, zeolites can bind certain cations more preferably than others, even when the preferred cation is present at much lower concentration than other cations in the fluid surrounding the zeolite. Zeolites have the ability to lose and absorb water without damage to their crystal structures.

The structural Si:Al ratio differs between zeolite types. High-aluminum zeolites have a high negative charge and thus a high number of exchangeable cations, and a high water absorption capacity. They are termed ‘hydrophilic’ zeolites, while low-aluminum zeolites are called ‘hydrophobic’ and are able to adsorb hydrophobic molecules.

In order to achieve a high ion adsorption per gram zeolite, it is desirable to use a zeolite with a low ratio of Si:Al resulting in a high negative charge and thus a high ion-exchange capacity.

When binding potassium ions onto a zeolite, a counter-ion is released from the zeolite and enters the fluid. It is an advantage to use a zeolite containing sodium ions as the extra-framework counter-ion, as release of a sodium ion for each bound potassium ion will not significantly alter the sodium ion concentration of the dialysis fluid, which is already high. Alternatively, the zeolite may contain calcium or magnesium ions, or any mixture of sodium, calcium and magnesium ion as counter-ions.

We discovered that several zeolites in Na⁺-form, i.e. having sodium ions as extra-framework ions, are able to specifically bind potassium ions and release sodium ions even if the concentration of sodium is high and that of potassium is low in the dialysis fluid. Zeolites with high aluminum content are the most efficient ion exchangers as one extra-framework ion is bound for each aluminum atom in the zeolite framework. We discovered that such zeolites have a high potassium ion binding capacity in dialysis fluids, and can be used for binding potassium ions present at a concentration below 5 mM in dialysis fluid where sodium ion concentration is high (133 mM) and low concentrations of calcium (1.75 mM) and magnesium ions (0.25 mM) are present.

During experimentation with such a zeolite, it was surprisingly found that the zeolite caused an increase of the pH of the solution, in which it was incubated.

Increased pH should be avoided in a peritoneal dialysis fluid for several reasons, one being that calcium tends to precipitate as calcium carbonate at increased pH. It seems that such increase in pH caused by the zeolite has been overlooked in the prior art, which may be due to the fact that the catalytic conversion of urea results in an increase of pH as reported in the above-mentioned document GB1484642A, which may have masked the increase caused by the zeolite.

The propensity of zeolites, especially hydrophilic ones with a high aluminum content to raise the pH of aqueous solutions, may be due to their strong affinity for positively charged ions. In water and dilute salt solutions this leads to the capture of H₃O⁺, an ion that is always present in aqueous solutions in varying amounts depending on the pH of the solution. When H₃O⁺ is bound by the zeolite, the balance of the equilibrium:

H₂O [symbol] H₃O⁺+OH⁻

is shifted to the right, and an excess of OH⁻ ions leads to an increase in the pH of the solution.

We found that high aluminum zeolites alter the pH of dialysis fluid from the physiological pH of 7.4 up to around 9. The pH of recirculating dialysis fluid may not deviate significantly in the alkaline direction, in order to retain patient safety and well-being and prevent precipitation of calcium carbonate. This makes high-aluminum zeolites unsuitable for regeneration of dialysis fluid.

We found that a zeolite with intermediate aluminum content (Si:Al ratio 5.5:1), gives adequate potassium ion binding capacity in dialysis fluid without dramatic effects on pH. This discovery solves the problem of pH effects on the fluid caused by the high affinity for H₃O⁺ of high-aluminum zeolites.

To decrease the effect of high-aluminum zeolite on dialysis fluid pH, the zeolite can be titrated by acids, so that enough H₃O⁺ is added to the solution contacting the zeolite to counteract the alkalinization caused by uptake of H₃O⁺ by the zeolite. In this way, a stable neutral pH in the solution can be achieved. When the titrated zeolite is subsequently transferred to a fresh dialysis fluid containing K⁺ ions, the pH is kept stable at the expense of somewhat lower K⁺ binding capacity.

Another potassium adsorbent, which may be used is a cation exchange resin made from sodium polystyrene sulfonate USP, which is sold under the tradename AMBERLITE™ IRP 69. This potassium adsorbent is similar to oral potassium adsorbents, such as RESONIUM. The exchange resin IRP69 is loaded with sodium ions, which are released in exchange of potassium ions.

IRP 69 is a strong cation exchanger resin consisting of a sulfonated copolymer of styrene and divinylbenzene. The sulfone ligand strongly attracts positive ions, and can exchange one positive ion for another. IRP69 is supplied with sodium as the exchangeable cation, and can bind other cations, such as potassium ions, during release of sodium ions. IRP69 is a pharmaceutical grade resin of small particle size with a total potassium exchange capacity of 110-135 mg/g. The product is mainly used as an oral potassium ion binder and as a drug carrier for sustained release applications, taste masking and drug stabilization.

However, the potassium adsorbent, such as IRP 69, also adsorbs calcium ions and magnesium ions, which is a drawback. However, if only small amounts of peritoneal dialysis fluid is used per day, such as below 3-5 liters, the removal of calcium and magnesium ions may be tolerated by the body and most of it being replenished by the food. Alternatively, calcium and magnesium can be added to the food in sufficient amounts.

In another embodiment, the potassium adsorbent, such as IRP69, is used and removes almost all potassium, calcium and magnesium ions, and releases sodium ions in their place. However, after the removal, the system or device may add calcium and magnesium ions, for substantially compensating for the loss. This may be in addition to oral supply or as an alternative.

In a hemodialysis system using regeneration, the fluid volume may be larger than 3 liters, up to for example 10 liters. In such a system, adsorptive potassium removal will not be necessary, because a dialysis fluid with zero potassium will remove a sufficient amount of potassium. For certain hyperkalemic patients, addition of a small amount of the potassium adsorbent may be desired, and such small amount will be tolerated, possibly after food complementation of magnesium and calcium. Alternatively or additionally, the hemodialysis device may add calcium and magnesium ions, for substantially compensating for the loss.

### Examples

### Potassium Ion Binding Capacity of Zeolites

1 g of zeolite was pre-equilibrated during about 20 hours with 200 mL equilibration fluid containing 133 mM Na⁺, 1.75 mM Ca²⁺, 0.25 mM Mg²⁺, 100 mM Cl⁻, 10 mM lactate, 25 mM bicarbonate, 75 mM glucose. The fluid was decanted and 300 mL of a PD fluid containing 133 mM Na⁺, 1.75 mM Ca²⁺, 0.25 mM Mg²⁺, 105 mM Cl⁻, 10 mM lactate, 25 mM bicarbonate, 75 mM glucose and 5 mM K⁺ was added. The flasks were incubated on magnetic stirrers. Samples were taken after 30 minutes and analyzed for residual K⁺ content. The specific K⁺-binding capacity was calculated. Zeolite X (Zeolum F-9) with a high aluminum content (Si:Al ratio about 1.5:1) bound 0.57 mmol K⁺/g, and Zeolite Y (HSZ-320NAA) with an ‘intermediate’ aluminum content (Si:Al ratio 5.5:1) bound 0.36 mmol K⁺/g.

Effect of Zeolites on the pH of Dialysis Fluid

About 0.5 g zeolite was suspended in 20 mL equilibration fluid containing 133 mM Na⁺, 1.75 mM Ca²⁺, 0.25 mM Mg²⁺, 100 mM Cl⁻, 10 mM lactate, 25 mM bicarbonate, 75 mM glucose. pH was measured 5 minutes after the addition, and compared with the pH in 20 mL PD fluid without zeolite. Zeolite X (Zeolum F-9) with a high aluminum content increased the pH by 1.49 units, while Zeolite Y (HSZ-320NAA) with an ‘intermediate’ aluminum content (Si:Al ratio 5.5:1) increased the pH by 0.17 units.

Effect of Zeolites on the pH of Dialysis Fluid after Pre-Equilibration

pH was measured in the samples of example 1 where the zeolite had first been pre-equilibrated with an equilibration fluid containing all PD fluid components but no K⁺ ions, and subsequently incubated with the same fluid with added K⁺ ions. The pH of the samples incubated with zeolite was compared with the pH of 200 mL of the same fluid incubated on a magnetic stirrer without zeolite. Zeolite X (Zeolum F-9) with a high aluminum content increased the pH by 0.74 units, while Zeolite Y (HSZ-320NAA) with an ‘intermediate’ aluminum content (Si:Al ratio 5.5:1) increased the pH by 0.17 units.

Titration of Zeolite with High Aluminum Content

20 g of Zeolite X (Zeolum F-9HA) in pellet form was washed 4 times with 200-400 mL equilibration fluid containing 133 mM Na⁺, 1.75 mM Ca²⁺, 0.25 mM Mg²⁺, 95 mM Cl⁻, 40 mM lactate and 75 mM glucose. The last wash was incubated overnight. The pH of the solution was 9.1 when titration with 1 M hydrochloric acid was started. After gradual addition of about 2.3 mL 1 M acid the pH stabilized at 7.1. After storage for 7 days the pH was 7.3. When comparing the K⁺ binding capacity and pH effect of titrated and untitrated Zeolite X (Zeolum F-9HA) in pellet form in a continuous flow-column, the binding capacity was 0.37 mmol/g for untitrated zeolite and 0.24 mmol/g for titrated zeolite. The titrated zeolite had no significant effect on pH of the fluid during 80 min of flow, whereas the untitrated zeolite initially raised the pH by 2.66 units, and after 2.5 hours of flow the pH increase was 2.03 units.

Compensation of pH Effect of Zeolite with High Aluminum Content by Using Equilibration Fluid with a Low pH

From data on the amount of acid per gram of zeolite needed to titrate Zeolite X (Zeolum F-9HA) to pH 7.4, obtained from the previous example, it was concluded that approximately 0.6 mL of 1 M HCl would be necessary to titrate 5 g of Zeolite X pellets to pH 7.4. This amount of HCl was added to 200 mL of an equilibration fluid containing 133 mM Na⁺, 1.75 mM Ca²⁺, 0.25 mM Mg²⁺, 95 mM Cl⁻, 40 mM lactate and 75 mM glucose. The pH of the fluid was set to 7.4 prior to the addition of HCl, and was lowered to 4.9 after the addition. 5 g of Zeolite X pellets were added to the fluid. The pH of the solution rose slowly to 7.6 during 26 hours, whereafter another 0.1 mL of HCl was added and pH decreased to 6.3. After 3 days, pH was 7.5 and another 0.1 mL of HCl was added. After 2 days the pH was 7.0. Thus, the titrating acid does not have to be added gradually to the zeolite, but can be pre-added to the fluid before addition of the zeolite, whereafter the pH will slowly adjust to around 7.4.

The K⁺ binding capacity of the zeolite after equilibration in acid-containing dialysis fluid was tested as described in the first example, and found to be 0.14 mmol/g zeolite after 30 min incubation, increasing to 0.21 mmol/g after 4 h incubation. The pH of the K⁺ containing test dialysis fluid did not change during the 4 h incubation.

Comparison of potassium ion binding using a sulphonated styrene-divinylbenzene copolymer strong cation exchanger Amberlite IRP69 and two zeolites: Zeolite X and Zeolite Y.

5 g of IRP69 resin was swelled in 50 ml 135 mM NaCl solution overnight. The swelled resin was filtered and washed with 150 ml 135 mM NaCl. 5 g of dry Zeolite X (Zeolum F-9, Si:Al ratio 1.5:1) and Zeolite Y (HSZ-320NAD1A, Si:Al ratio 5.5:1) pellets, pretreated as described in the example ‘Titration of zeolite with high aluminum content’ and dried in 80° C., and 5 g swelled IRP69, were put in 250 ml Erlenmeyer flasks. To each flask, 200 ml PD fluid, containing 133 mM Na⁺, 1.75 mM Ca2⁺, 0.25 mM Mg2⁺, 105 mM Cl—, 40 mM lactate, 75 mM glucose and 5 mM K⁺, pH 7.4 was added and the flasks were incubated on an orbital shaker for 1 hour. Samples were taken after 1, 10 20 and 60 minutes and analyzed for remaining K⁺ concentration. After 60 min, the K⁺ concentration in all flasks was between 2.3-2.5 mM, corresponding to specific K⁺ binding of 0.1 mmol IC/g binder for both zeolites and the IRP69 resin. Samples at different time-points from the IRP69 resin flask all contained the same amount of residual K⁺ as the 60 min sample, indicating that the K⁺ binding was a very rapid process. In contrast, the K⁺ concentration in the zeolite flasks decreased gradually, being 5 mM after 1 min, 3.7 after 10 min, 3 mM after 20 mM and 2.4 after 60 min, indicating a slower K⁺ binding.

K⁺ Binding to Sulphonated Styrene-Divinylbenzene Copolymer Cation Exchanger Resin Amberlite IRP69 in a Continuous Flow System.

10 g (dry weight) of IRP69 resin, pre-swelled and washed as described in example 1, was suspended in 135 mM NaCl and transferred into a plexiglass column of 2.2 cm diameter. The resin was allowed to settle in the column. PD fluid with the composition as described in example 1 was pumped from the bottom up through the column with a flow rate of 7 mL/min. Samples were taken at the outlet of the column. During the first 30 min the potassium concentration in the outlet was below 1.5 mM, and gradually increased to 3 mM after 70 min and 4.2 mM after 100 min. The specific potassium ion binding during 100 minutes was 0.2 mmol/g of dry IRP69 resin.

K⁺ Binding to Sulphonated Styrene-Divinylbenzene Copolymer Cation Exchanger Resin Amberlite IRP69 in a Continuous Flow System.

The experiment described in example 2 was repeated, but the column was loaded with 19 g dry IRP69 resin. PD fluid containing all components as described in example 1 except for KCl was pumped through column, until the resin was wetted and a stable back pressure was obtained. The experiment was started by exchanging the PD fluid without KCl to PD fluid containing 5 mM KCl. Fluid was pumped through the column at 10 ml/min flow rate and samples were taken at the outlet of the column. K⁺ concentration in the outlet fluid was below 1.5 mM during the first 60 min, and after that rose gradually until it reached the same concentration as the inlet fluid after 180 min. The specific potassium ion binding was 0.2 mmol/g of dry IRP69 resin.

In the claims, the term “comprises/comprising” does not exclude the presence of other elements or steps. Furthermore, although individually listed, a plurality of means, elements or method steps may be implemented by e.g. a single unit. Additionally, although individual features may be included in different claims or embodiments, these may possibly advantageously be combined, and the inclusion in different claims does not imply that a combination of features is not feasible and/or advantageous. In addition, singular references do not exclude a plurality. The terms “a”, “an”, “first”, “second” etc. do not preclude a plurality. Reference signs in the claims are provided merely as a clarifying example and shall not be construed as limiting the scope of the claims in any way.

Although the present invention has been described above with reference to specific embodiment and experiments, it is not intended to be limited to the specific form set forth herein. Rather, the invention is limited only by the accompanying claims and, other embodiments than those specified above are equally possible within the scope of these appended claims.

1. A method of adsorption of ions from a peritoneal dialysis fluid, which has been removed from the peritoneal cavity of a patient performing peritoneal dialysis for subsequent recirculation and re-introduction into the peritoneal cavity of the patient, or from a hemo-dialysis fluid which has been removed from a dialyzer for subsequent recirculation and re-introduction into the dialyzer;
further comprising the step of:
contacting the removed dialysis fluid with a potassium adsorbent for adsorption of potassium ions.

2. The method according to claim 1, wherein the potassium adsorbent is an ion exchange material, which adsorbs potassium ions and releases sodium ions in their place.

3. The method according to claim 1, wherein the amount of dialysis fluid is less than 10 liters and that the dialysis fluid is discarded at least once per day.

4. The method according to claim 3, wherein the amount of dialysis fluid is less than 5 liters, such as less than 3 liters.

5. The method according to claim 1, wherein the potassium adsorbent is a sodium polystyrene sulfonate ion exchange material.

6. The method according claim 1, in which additional binding of calcium ions and magnesium ions by the adsorbent is compensated.

7. The method according to claim 6, wherein the compensation is performed by oral intake.

8. The method according to claim 6, wherein the compensation is performed by addition of calcium ions and magnesium ions to the recirculated fluid, downstreams of the ion exchanger.

9. The method according to claim 1, wherein the potassium adsorbent is a zeolite, which has been pre-equilibrated with an equilibration fluid comprising at least one of: Na⁺, Ca²⁺, Mg²⁺, Cl⁻, lactate, bicarbonate and glucose, which equilibration fluid lacks K⁺.

10. The method according to claim 9, wherein said equilibration fluid contains: Na⁺, Ca²⁺, Mg²⁺, Cl⁻ and lactate, in substantially the same concentration as in peritoneal dialysis fluid.

11. The method according to claim 9, wherein said zeolite has a Si:Al ratio which is larger than 5:1.

12. The method according to claim 9, wherein said zeolite has a Si:Al ratio which is larger than 1:1 and wherein the zeolite during or after the pre-equilibration is titrated to near neutral pH using acid.

13. The method according to claim 12, wherein the acid is added to the equilibration fluid so that said fluid before or during the pre-equilibration has a pH of less than 5.0.

14. A potassium adsorbent arranged in a regeneration circuit for a peritoneal dialysis fluid, which has been removed from the peritoneal cavity of a patient performing peritoneal dialysis for subsequent recirculation and re-introduction into the peritoneal cavity of the patient, or from a hemo-dialysis fluid which has been removed from a dialyzer for subsequent recirculation and re-introduction into the dialyzer.

15. The potassium adsorbent of claim 14, comprising a sodium polystyrene sulfonate ion exchange material.

16. The potassium adsorbent of claim 14, comprising a is a zeolite, which has been pre-equilibrated with an equilibration fluid comprising at least one of: Na⁺, Ca²⁺, Mg²⁺, Cl⁻, lactate, bicarbonate and glucose, which equilibration fluid lacks K⁺.
